(19) United States
(12) Patent Application Publication
(10) Pub. No.: US 2025/0352183 A1
(43) Pub. Date: Nov. 20, 2025

(54) MEDICAL INFORMATION PROCESSING DEVICE, ULTRASONIC DIAGNOSIS DEVICE, AND NON-TRANSITORY COMPUTER READABLE MEDIUM

(71) Applicants: CANON MEDICAL SYSTEMS CORPORATION, Tochigi (JP); Canon Kabushiki Kaisha, Tokyo (JP)

(72) Inventors: Masashi USUMURA, Nasushiobara (JP); Shoya SASAKI, Yokohama-shi (JP)

(73) Assignees: CANON MEDICAL SYSTEMS CORPORATION, Tochigi (JP); Canon Kabushiki Kaisha, Tokyo (JP)

(21) Appl. No.: 19/208,794

(22) Filed: May 15, 2025

(30) Foreign Application Priority Data
May 16, 2024 (JP) 2024-080188

(51) Int. Cl. A61B 8/00; A61B 8/06

(52) U.S. Cl. CPC A61B 8/5207; A61B 8/06; A61B 8/488

(57) ABSTRACT

A medical information processing device provided in one aspect of the present invention includes a processing circuit. The processing circuit acquires a plurality of pieces of first ultrasonic data obtained based on a result of an ultrasonic scan of a subject, applies a transformation to the pieces of first ultrasonic data to generate a plurality of pieces of second ultrasonic data characterized by a parameter other than time, continuously extracts signal components representing an object from the pieces of second ultrasonic data, and outputs third ultrasonic data, based on the continuously extracted signal components representing the object.

### CROSS-REFERENCE TO RELATED APPLICATIONS

This application is based upon and claims the benefit of priority from Japanese Patent Application No. 2024-080188, filed on May 16, 2024; the entire contents of which are incorporated herein by reference.

### FIELD

Embodiments disclosed herein relate to a medical information processing device, an ultrasonic diagnosis device, and a non-transitory computer readable medium.

### BACKGROUND

Ultrasonic diagnosis devices are widely used to observe and diagnose blood flow in the body. An ultrasonic diagnosis device generates and displays blood flow information from reflected ultrasonic waves using a Doppler method based on a Doppler effect. The blood flow information generated and displayed by the ultrasonic diagnosis device includes color Doppler images, Doppler waveforms (Doppler spectrum), and the like.

The color Doppler images are captured by a color flow mapping (CFM) method. In the CFM method, ultrasonic waves are transmitted and received a plurality of times on a plurality of scanning lines. Then, by applying a moving target indicator (MTI) filter to data sequences at the same position, signals derived from stationary or slowly moving tissue (clutter signals) are suppressed and signals derived from the blood flow are extracted. In the CFM method, the blood flow information such as a velocity value, a variance value, and a power value of the blood flow is estimated from these blood flow signals, and the distribution of the estimated results is displayed as a Doppler image.

It is known that the resolution in B-mode data and Doppler data decreases by the point spread function (PSF), which is determined by the wavelength of the transmission ultrasonic waves and the transmission/reception aperture width. There are solutions such as increasing the frequency of the transmission ultrasonic waves, but since there is a limitation in the frequency bandwidth of a probe, the resolution of the images that can be acquired is limited.

“Fast super resolution ultrasonic imaging using the erythrocytes” describes generating blood flow data with improved resolution by extracting and integrating portions with high signal values (amplitude values) from a plurality of pieces of blood flow data that are continuous in a time direction. More specifically, “Fast super resolution ultrasonic imaging using the erythrocytes” describes the use of the fact that the amplitude distribution characteristic of a speckle pattern in blood flow data can be substantially approximated by a probability distribution called the Rayleigh distribution. In this way, a high-luminance, spatially sparse signal with a small probability of occurrence but a large signal value (amplitude value) is extracted from each piece of blood flow data and the obtained signals are integrated.

However, in the method described in “Fast super resolution ultrasonic imaging using the erythrocytes”, if the number of frames used to extract an object is small, the object is extracted discretely (discontinuously) and high-resolution blood flow data (ultrasonic data) cannot be obtained. On the other hand, if a large number of frames are used, the load on the arithmetic processing increases, resulting in poor responsiveness.

### BRIEF DESCRIPTION OF THE DRAWINGS

FIG. 1 is a block diagram illustrating a structure of an ultrasonic diagnosis device according to an embodiment;

FIG. 2 is a diagram for describing a Rayleigh distribution;

FIG. 3 is a flowchart expressing a procedure of a process of the ultrasonic diagnosis device according to the embodiment;

FIG. 4 is a schematic diagram for describing a process to be performed by the ultrasonic diagnosis device according to a first embodiment;

FIG. 5 is a diagram for describing transformation for generating second ultrasonic data in the first embodiment;

FIG. 6 is a diagram illustrating an example of a kernel used for a process at step S500 in FIG. 3;

FIG. 7A is a diagram illustrating one example of the second ultrasonic data corresponding to an object of extraction of a signal component;

FIG. 7B is a diagram expressing a result of extracting an object when a kernel according to a comparative example is used;

FIG. 7C is a diagram expressing a result of extracting the object when the kernel according to the embodiment is used; and

FIG. 8 is a diagram for describing transformation for generating the second ultrasonic data in a second embodiment.

### DETAILED DESCRIPTION

A medical information processing device provided in one aspect of the present invention includes a processing circuit. The processing circuit acquires a plurality of pieces of first ultrasonic data obtained based on a result of an ultrasonic scan of a subject, applies a transformation to the pieces of first ultrasonic data to generate a plurality of pieces of second ultrasonic data characterized by a parameter other than time, continuously extracts signal components representing an object from the pieces of second ultrasonic data, and outputs third ultrasonic data, based on the continuously extracted signal components representing the object.

### First Embodiment

One embodiment of a medical information processing device, an ultrasonic diagnosis device, and a computer program is described below in detail with reference to the drawings.

The ultrasonic diagnosis device according to this embodiment causes an ultrasonic probe to perform an ultrasonic scan and collects a plurality of pieces of frame data that are obtained by the ultrasonic scan and that are continuous in a time direction (a plurality of pieces of frame data within a predetermined time). The frame data is collected at a predetermined frame rate by performing the ultrasonic scan. The frame data refers to any of reception data, measurement data, blood flow data, and tissue data. The reception data is, for example, a reception signal of ultrasonic waves received by the ultrasonic probe (for example, CH data). The measurement data is data (for example, IQ data) obtained by performing delay and sum and quadrature detection processing on the reception signal of the ultrasonic waves. The blood flow data is data (for example, power signal data) where the information of the measurement data that is derived from the blood flow is extracted or emphasized. The tissue data is data (for example, B-mode data) where the information that is derived from the tissue is extracted or emphasized.

The measurement data includes, for example, tissue-derived information (tissue signal component (clutter)) and blood flow-derived information (blood flow signal component). The blood flow-derived information may include not just information derived from blood, but also information derived from a contrast medium in the blood. The blood flow data is data where the blood flow-derived information is extracted or emphasized, and includes the velocity value, the variance value, and the power value of the blood flow.

The extraction of the blood flow-derived information corresponds to, for example, an operation of extracting the blood flow signal component from the measurement data. The emphasis of the blood flow-derived information corresponds to, for example, an operation of making the blood flow signal component stand out relative to the tissue signal component. The blood flow data may be obtained by a process of extracting or emphasizing the blood flow-derived information, or by a process of removing or reducing the tissue-derived information.

In other words, the ultrasonic diagnosis device acquires the pieces of first ultrasonic data obtained based on the result of the ultrasonic scan of the subject. Subsequently, the pieces of first ultrasonic data are transformed to generate the second ultrasonic data characterized by the parameter other than time. One example of the parameter other than time is frequency. The ultrasonic diagnosis device continuously extracts the signal components representing the object from the pieces of second ultrasonic data. The object includes, for example, any of blood, an in-body tissue, and a contrast medium.

The ultrasonic diagnosis device outputs the third ultrasonic data on the basis of the continuously extracted signal components representing the object. The third ultrasonic data may be any of synthesis data obtained by synthesizing the continuously extracted signal components representing the object and the second ultrasonic data, correction data obtained by correcting the first ultrasonic data or the second ultrasonic data, based on the continuously extracted signal components representing the object, and data including the continuously extracted signal components representing the object.

By this ultrasonic diagnosis device according to this embodiment, the object can be extracted continuously even if the number of frames used to extract the object is small because the object is extracted using the second ultrasonic data characterized by the parameter other than time. Thus, highly responsive and high-resolution ultrasonic data (high-resolution data) can be obtained. The term “high resolution” here includes both cases where the resolution is increased by increasing the pixel density of the image and where the spatial resolution is increased by extracting peak signals, although the pixel density of the image is not increased.

Although the example of applying the present disclosure to the ultrasonic diagnosis device has been described so far, the present disclosure may be applied to modalities (medical information processing devices) other than the ultrasonic diagnosis device. For example, the present disclosure can be applied to medical information processing devices such as workstations and servers that acquire ultrasonic data obtained based on the results of ultrasonic scans of subjects.

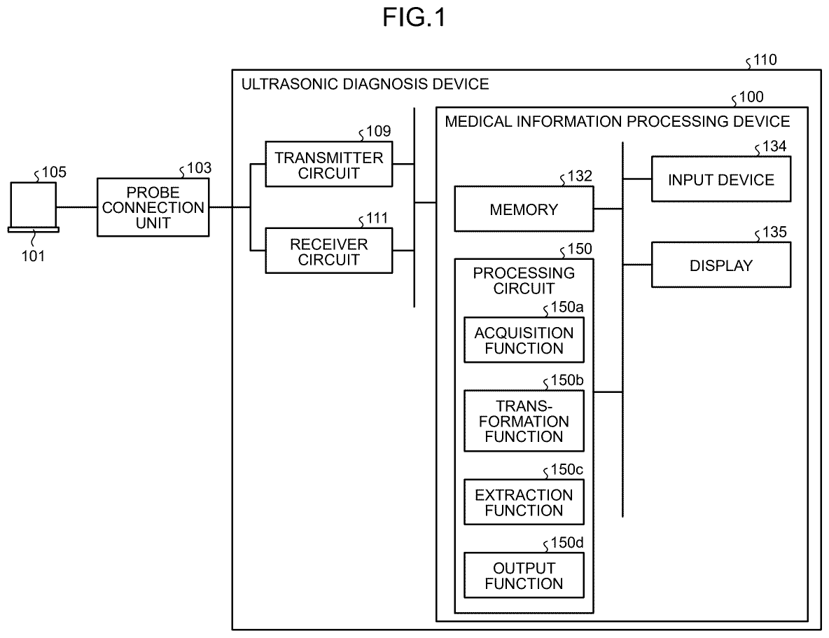

FIG. 1 is a block diagram illustrating a structure of the ultrasonic diagnosis device according to the embodiment. An ultrasonic diagnosis device 110 is a device that generates ultrasonic data on the basis of reception signals (reflection wave signals) received from an ultrasonic probe 105. The ultrasonic diagnosis device 110 illustrated in FIG. 1 can generate two-dimensional ultrasonic data on the basis of two-dimensional reception signals and generate three-dimensional ultrasonic data on the basis of three-dimensional reception signals. However, the embodiment is applicable even when the ultrasonic diagnosis device 110 is a device dedicated to the two-dimensional data. The ultrasonic diagnosis device 110 includes a transmitter circuit 109, a receiver circuit 111, and a medical information processing device 100.

The ultrasonic probe 105 is, for example, an electronic scanning-type probe and has a plurality of transducer elements 101 arranged one-dimensionally or two-dimensionally at its tip. The transducer element 101 is a piezoelectric element (electromechanical conversion element) that performs mutual conversion between electrical signals (voltage pulse signals) and ultrasonic waves (acoustic waves). The ultrasonic probe 105 transmits ultrasonic waves from the transducer elements 101 to the subject, and receives the reflection ultrasonic waves from the subject by the transducer elements 101. The reflection acoustic waves reflect differences in acoustic impedance within the subject. When the transmitted ultrasonic pulse is reflected on a moving blood flow or on a surface of a heart wall or the like, the reflection ultrasonic waves are subject to frequency shift depending on a velocity signal component relative to an ultrasonic wave transmission direction of a moving object due to the Doppler effect.

A probe connection unit 103 connects the ultrasonic probe 105 and transmits and receives ultrasonic waves to and from the ultrasonic probe 105. The connection of the ultrasonic probe 105 by the probe connection unit 103 may or may not use a wire. In the case of using the wire, the probe connection unit 103 includes a connector part (receptacle) to which a connector (plug) of the ultrasonic probe 105 is connected. In the case of not using the wire, a communication unit that performs wireless communication with the ultrasonic probe 105 is provided.

The transmitter circuit 109 is a transmission unit that outputs pulse signals (driving signals) to the transducer elements 101. By applying the pulse signals with time differences to the transducer elements 101, ultrasonic waves with different delay times are transmitted from the transducer elements 101 to form a transmission ultrasonic beam. The direction and focus of the transmission ultrasonic beam can be controlled by selectively changing the transducer element 101 to which the pulse signal is applied or by changing the delay time (application timing) of the pulse signal. By sequentially changing the direction and focus of the transmission ultrasonic beam, an observation region inside the subject is scanned. By changing the delay time of the pulse signal, a transmission ultrasonic beam may be formed that is a plane wave (a focus is far away) or a diffuse wave (a focus point is the opposite of the ultrasonic transmission direction with respect to the transducer elements 101). Alternatively, one transducer element or some of the transducer elements 101 may be used to form the transmission ultrasonic beam. The transmitter circuit 109 transmits a pulse signal with a predetermined driving waveform to the transducer element 101 to generate the transmission ultrasonic wave with a predetermined transmission waveform at the transducer element 101.

The receiver circuit 111 is a reception unit that inputs the electrical signal output from the transducer element 101, which has received the reflection ultrasonic wave, as the reception signal. The reception signal is input to a processing circuit 150. In this embodiment, both an analog signal output from the transducer element 101 and digital data sampled (digitally converted) from the analog signal are referred to as the reception signal without any particular distinction. However, depending on the context, the reception signal may be described as reception data or measurement data for the purpose of explicitly indicating that the data is digital data.

The medical information processing device 100 is connected to the transmitter circuit 109 and the receiver circuit 111, processes signals received from the receiver circuit 111, and executes control of the transmitter circuit 109. The medical information processing device 100 includes the processing circuit 150, a memory 132, an input device 134, and a display 135.

The memory 132 includes a semiconductor memory element such as a random-access memory (RAM) or a flash memory, a hard disk, an optical disc, or the like. The memory 132 is a memory that stores therein data such as image data for display generated by the processing circuit 150. The memory 132 can also store therein the reception signal (reflection wave signal) output by the receiver circuit 111. In addition, the memory 132 stores therein control programs for performing ultrasonic transmission and reception, image processing, and display processing, as well as diagnostic information (for example, patient ID, physician's findings, etc.), diagnostic protocols, various body marks, and other data as needed.

The input device 134 receives various instructions and information input from an operator. The input device 134 includes an input interface device such as a mouse, a keyboard, buttons, or a trackball.

The display 135 displays a graphical user interface (GUI) for receiving the input of imaging conditions and various images under the control of the processing circuit 150. The display 135 includes, for example, a display interface device such as a liquid crystal display.

The processing circuit 150 controls each part of the ultrasonic diagnosis device 110, thereby controlling the entire ultrasonic diagnosis device 110. In FIG. 1, the processing circuit 150 is described as being realized alone, but the processing circuit 150 may alternatively be realized by combining a number of independent processors. Alternatively, specific functions may be formed of dedicated independent circuits, such as an application specific integrated circuit (ASIC).

The term “processor” used in the above description means, for example, a circuit such as a central processing unit (CPU), a graphical processing unit (GPU), an application specific integrated circuit (ASIC), a programmable logic device (for example, simple programmable logic device (SPLD)), a complex programmable logic device (CPLD), or a field programmable gate array (FPGA). The processor reads out and executes a computer program saved in the memory 132 to realize the function.

The processing circuit 150 causes the ultrasonic probe 105 to perform an ultrasonic scan and collects a plurality of pieces of frame data that are obtained by the ultrasonic scan and that are continuous in the time direction (a plurality of pieces of frame data within a predetermined time). The processing circuit 150 performs delay and sum and quadrature detection processing on the reception signal (CH data) collected through the receiver circuit 111. The delay and sum processing is a process of adding up the reception signals of the transducer elements 101 with the delay time and weight varied for each transducer element 101, and is also known as delay and sum (DAS) beam forming. The quadrature detection processing is a process of converting the reception signal into an in-phase signal and a quadrature signal (IQ data (measurement data)) in a baseband range. In addition, the reception signal may be subjected to a process using adaptive beam forming, model-based processing, machine learning, or the like.

The processing circuit 150 may also estimate the amount of tissue displacement due to body motion of the subject or the like between the pieces of frame data, and correct each piece of frame data on the basis of the results of estimation. Specifically, the processing circuit 150 calculates the amount of tissue displacement due to body motion or the like between the frames from the pieces of frame data. The processing circuit 150 corrects the frame data on the basis of the calculated amount of displacement.

Moreover, the processing circuit 150 performs an envelope detection process, a logarithmic compression process, or the like so as to generate B-mode data (data in which tissue-derived information is extracted or emphasized) representing, in luminance, the signal intensity at each point in the observation region. The processing circuit 150 also generates blood flow data (power signal data) in which the blood flow-derived information is extracted or emphasized in the measurement data.

For example, the processing circuit 150 applies a moving target indicator (MTI) filter to the pieces of frame data. This reduces the information derived from tissue that is stationary or has little movement between the frames (tissue signal components (clutter)), and extracts the blood flow-derived information (blood flow signal components). As the MTI filter, a filter with a fixed filter coefficient, such as a Butterworth-type infinite impulse response (IIR) filter or a polynomial regression filter may be used. The MTI filter may be an adaptive filter that varies its coefficient according to the input signal using eigenvalue or singular value decomposition, or the like.

The processing circuit 150 may also decompose the frame data into a plurality of bases by eigenvalue or singular value decomposition, or the like, remove the tissue-derived information by extracting a specific base, and extract the blood flow-derived information. The processing circuit 150 may obtain a velocity vector for each set of coordinates in the reception signal data and obtain a blood flow vector representing the size and direction of the blood flow in accordance with a vector Doppler method, a speckle tracking method, a vector flow mapping method, or the like. In addition to the methods given here, any method that can extract or emphasize the blood flow-derived information included in the frame data (reception data and measurement data) or remove or reduce the tissue-derived information may be used.

Furthermore, the processing circuit 150 reads out and executes a computer program stored in the memory 132 so as to activate an acquisition function 150a, a transformation function 150b, an extraction function 150c, and an output function 150d, thereby outputting high-resolution ultrasonic data.

Before the process of outputting the high-resolution ultrasonic data using the ultrasonic diagnosis device 110 configured as above is described, a method of generating high-resolution data according to conventional art (for example, the method described in “Fast super resolution ultrasonic imaging using the erythrocytes”) is described.

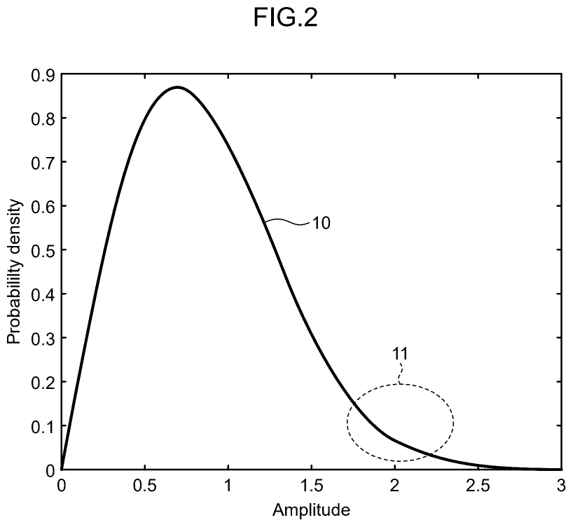

It is known that an amplitude distribution characteristic 10 of a speckle pattern of the blood flow data can be substantially approximated by a probability distribution called the Rayleigh distribution, as illustrated in FIG. 2. In the method of generating the high-resolution data in the conventional art, a high-luminance, spatially sparse signal (area 11 indicated in FIG. 2) with a small probability of occurrence but a large signal value (amplitude value) is extracted from a plurality of pieces of blood flow data corresponding to the respective pieces of frame data and by integrating this, ultrasonic data is generated.

Here, in the conventional method of generating the high-resolution data, the high-luminance, spatially sparse signal is extracted for each frame labeled at each time. On the other hand, in the method of generating the high-resolution data according to the embodiment, as will be explained below, the high-luminance, spatially sparse signal is extracted for each frame labeled by a parameter other than time, for example, frequency.

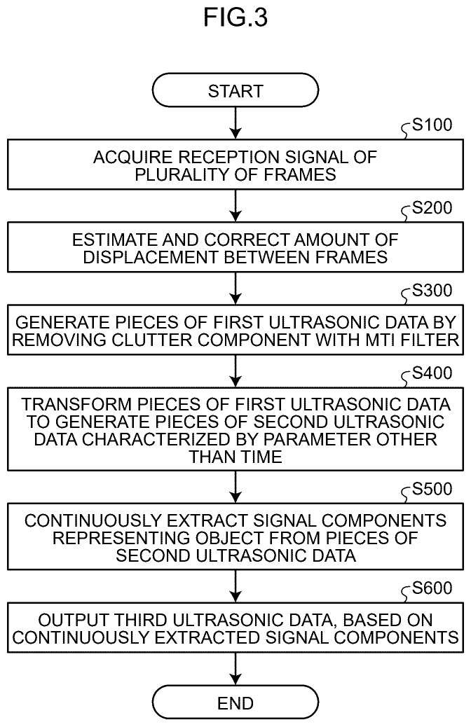

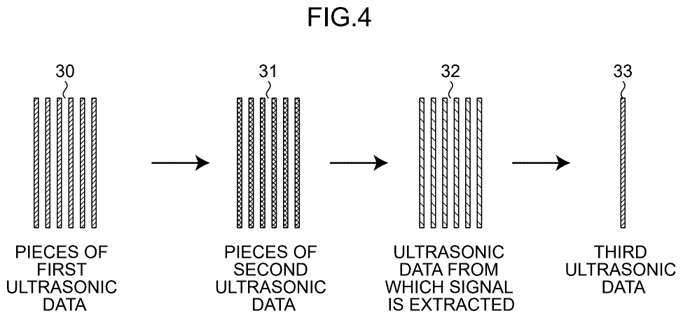

With reference to FIG. 3 and FIG. 4, the process to be performed by the medical information processing device 100 will be described below. The processing circuit 150 causes the acquisition function 150a to collect and acquire the frame data (reception signals, measurement data, etc.) that are obtained based on the results of the ultrasonic scan of the subject and that are continuous in the time direction as illustrated in FIG. 4 (step S100). The number of frames in the frame data when generating the high-resolution data is about the same as that when generating the general Doppler data. For example, the number of frames (packets) used to generate the Doppler data is about several dozen. On the other hand, the number of pieces of frame data to be collected by the acquisition function 150a is about several dozen to several hundred, which is much smaller than the number of pieces of frame data to be collected by the method according to the conventional art (about several thousand to several tens of thousands).

The processing circuit 150 estimates the amount of tissue displacement due to body motion of the subject or the like between the pieces of frame data, and corrects each piece of frame data on the basis of the result of estimation (step S200). Specifically, the processing circuit 150 calculates the amount of tissue displacement due to body motion or the like between the frames from the reception signals containing data sequences of the frames. The processing circuit 150 corrects the amount of displacement by moving the reception signals so that the positions thereof are aligned within the frame with the use of the calculated amount of displacement. Here, a reference frame for calculating the amount of displacement may be just one frame for the entire data sequences, the reference frame may be changed according to the position in the time direction (that is, a plurality of reference frames may be provided), or the previous frame in the adjacent frames may be used as the reference frame. The reference frame may be selected arbitrarily from the data sequences, and for example, any of the first frame, the middle frame, or the last frame may be used as the reference frame.

The processing circuit 150 applies an MTI filter to the pieces of frame data with the amount of displacement corrected. This reduces the information derived from tissue that is stationary or has little movement between the frames (tissue signal component (clutter)), and extracts the blood flow-derived information (blood flow signal component) (step S300). This allows the processing circuit 150 to remove the clutter component and generate the pieces of first ultrasonic data 30, as illustrated in FIG. 4. The pieces of first ultrasonic data 30 are pieces of ultrasonic data of a plurality of frames that are arranged along a time series. In this manner, the processing circuit 150 causes the acquisition function 150a to acquire the pieces of first ultrasonic data 30 obtained based on the results of the ultrasonic scan of the subject.

Subsequently, at step S400, the processing circuit 150 causes the transformation function 150b to apply a transformation to the pieces of first ultrasonic data 30 and generate a plurality of pieces of second ultrasonic data 31 characterized by the parameter other than time t. The first embodiment will describe the case where the transformation performed on the first ultrasonic data 30 at step S400 is (discrete) Fourier transformation and the parameter that is other than the time t is frequency.

Here, the meaning of applying a transformation to the pieces of first ultrasonic data 30 to generate the pieces of second ultrasonic data 31 characterized by the parameter other than the time t will be described. By generating the pieces of ultrasonic data characterized by the parameter other than the time t, the transformation function 150b can separate a blood flow image, for example, into a plurality of characteristic-separated images. Here, the characteristic-separated image is, for example, an image in which the following images are separated: an image where a flow velocity component in a slow velocity range is extracted and an image where a fast velocity range is extracted. The processing circuit 150 can separate the images for each flow velocity range, for example, by choosing the frequency as the parameter other than the time t.

As a specific processing method, the processing circuit 150 causes the transformation function 150b to perform discrete Fourier transformation on the pieces of first ultrasonic data 30 to generate the pieces of second ultrasonic data 31.

Specifically, the pieces of first ultrasonic data 30 are represented by F(t) as a function of the time t, and F(t) is expanded with a base obtained by analyzing and connecting a trigonometric function to a complex plane; then, the pieces of first ultrasonic data 30 can be expanded as shown in Expression (1) in which N represents the number of frames and an expansion coefficient f(x) is a function of a parameter x that is other than the time t. The expansion coefficient f(x) is used as the pieces of second ultrasonic data 31.

$$F ( t ) = {\sum }_{x = 0}^{N - 1} f ( x ) exp ( - i \frac{2 π tx}{N} ) \qquad ( 1 )$$

The pieces of second ultrasonic data 31, which are the expansion coefficient f(x) defined in this way, can be calculated from F(t), which is the pieces of first ultrasonic data 30, by using the following Expression (2).

$$f ( x ) = \frac{1}{N} {\sum }_{t = 0}^{N - 1} F ( t ) exp ( i \frac{2 π xt}{N} ) \qquad ( 2 )$$

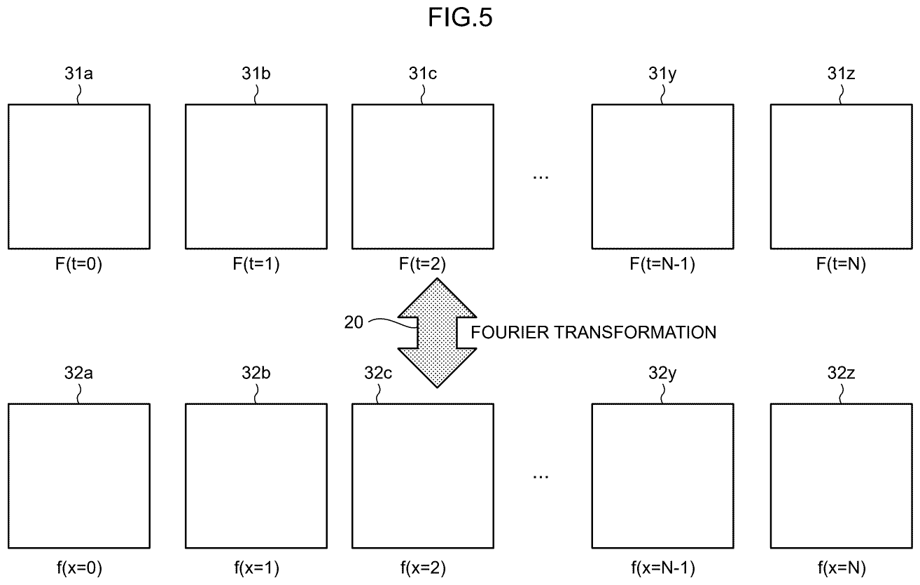

That is to say, as illustrated in FIG. 5, the transformation function 150b performs (discrete) Fourier transformation 20 on the pieces of first ultrasonic data 31a, 31b, 31c, . . . , 31y, 31z to calculate the pieces of second ultrasonic data 32a, 32b, 32c, . . . , 32y, 32z by using Expression (2). Here, the pieces of first ultrasonic data 31a, 31b, 31c, 31y, and 31z are the data in which the time t corresponds to t=0, t=1, t=2, t=N−1, and t=N, respectively, and the pieces of second ultrasonic data 32a, 32b, 32c, 32y, and 32z are the data in which the parameter x that is other than the time t corresponds to x=0, x=1, x=2, x=N−1, and x=N, respectively.

Referring back to FIG. 3, the extraction function 150c performs a process of continuously extracting the signal components representing the object for each parameter x other than the time t from the pieces of second ultrasonic data 31 generated at step S400 (step S500). The object here is the object whose resolution is increased, and includes, for example, at least one of blood, an in-body tissue, or a contrast medium. Continuously extracting the signal components representing the object means extracting the positions of signals (pixels) representing the object without an interval in the signal space or image space. For example, if the object is blood, the extraction function 150c extracts the positions of signals (pixels) representing blood without an interval in the signal space or image space. As a result, the extraction function 150c acquires the ultrasonic data 32 from which the signal is extracted, which is the extraction data representing the object, as illustrated in FIG. 4. The signal extraction at step S500 is performed for each parameter x other than the time t, that is, for each frequency in the first embodiment.

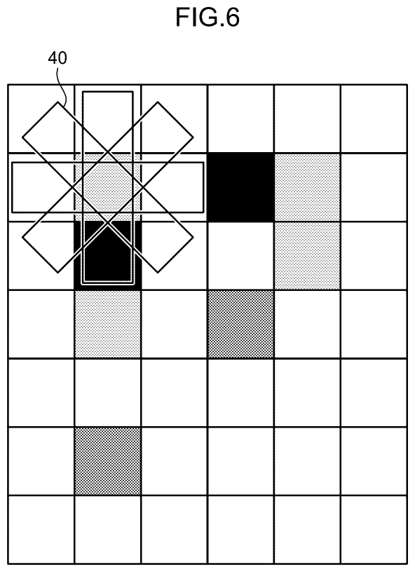

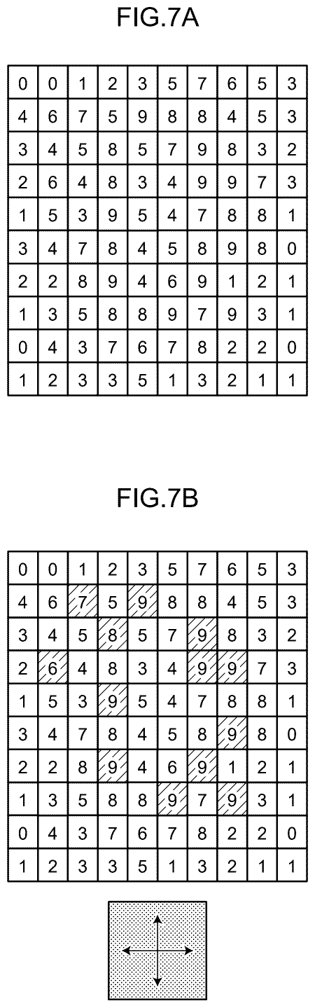

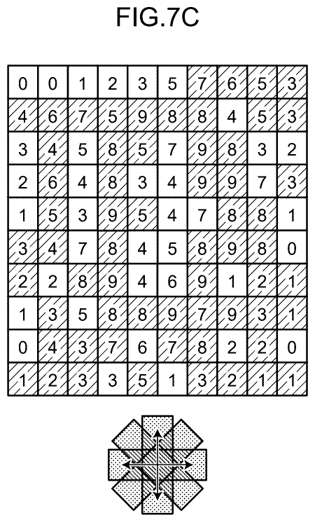

With reference to FIG. 6 and FIG. 7A to FIG. 7C, an example of a method for continuously extracting the signal components representing the object is described. For each parameter x other than the time t and for each of a plurality of attention positions included in the pieces of second ultrasonic data 31, based on a result of comparing a signal value at a position in predetermined attention direction and range with respect to the attention position and a signal value at the attention position, the extraction function 150c extracts a signal component at the attention position as the signal component representing the object. In one example, if the signal value at the attention position is more than or equal to the signal value at the position in the predetermined direction and range, the extraction function 150c extracts the signal component at the attention position as the signal component representing the object. The extraction function 150c extracts the position of signals (pixels) representing blood using a kernel 40 based on a ratio of 3 in height×1 in width, as illustrated in FIG. 6, for example. Here, for the convenience of explanation, the pixel value (luminance value) at the attention position and the pixel value at the position in the predetermined range are compared for each attention direction with respect to the attention position on the two-dimensional image in this example; however, the signal value at the attention position and the signal value at the position in the predetermined range may be compared one-dimensionally with respect to the second ultrasonic data 31. In other words, the signal components representing the object may be extracted without developing the second ultrasonic data 31 into an image.

Each square cell in FIG. 7A represents each pixel value (luminance value) of the image represented by the second ultrasonic data 31. When extracting the positions with the relatively high pixel values from the second ultrasonic data 31, for example, as illustrated in FIG. 7B, a kernel with total of 9 cells of 3×3 cells are used, including the cell at the attention position and eight cells adjacent to that attention position. In this case, when the pixel value at the attention position is higher than that of any of the eight adjacent cells, the processing circuit 150 extracts the attention position as the position of the pixel (signal) representing blood.

However, when the kernel illustrated in FIG. 7B is used, the object (blood vessel or the like) is extracted discretely and its structure is not adequately represented. On the other hand, if the kernel 40 illustrated in FIG. 6 is used, the object is extracted continuously as illustrated in FIG. 7C. Specifically, the kernel 40 includes three cells in a row, each cell corresponding to a single pixel. The pixel corresponding to the central cell is the attention position, and the pixels corresponding to the two cells adjacent to the central cell are the positions to be compared. More specifically, when the kernel 40 is set at the position illustrated in FIG. 6, the pixel value at the attention position is higher than the pixel values at the adjacent positions, and therefore, that attention position is extracted as the object.

In the above example of the kernel 40, the signal components representing the object are extracted in four directions of vertical, horizontal, diagonally upper right, and diagonally upper left directions, but the shape and size of the kernel are not limited to this example. Even when the pixel value at the attention position is equal to the pixel value at the position in the predetermined direction and range, the extraction function 150c may extract the attention position as the object.

In the above example, the object is extracted pixel by pixel, but the object may be continuously extracted in groups with a plurality of pixels. When the object is extracted in groups with the pixels, the average or integrated value of the pixels constituting the group may be used as the pixel value (signal value) of that group. In this case, the extraction function 150c extracts, for each group having the pixels, the signal component representing the object on the basis of the result of comparing the pixel values of another group at the position in the predetermined direction and range with the pixel values of the group at the attention position.

In another example, for each of the attention positions included in the first ultrasonic data 30, the extraction function 150c may compare the signal value (pixel value) at the attention position and the signal value (pixel value) at the position in the predetermined range for each attention direction with respect to the attention position, and based on the result of the comparison and a weight coefficient determined in advance for each attention direction, may weight the signal component at the attention position. For example, in the case where the four directions of vertical, horizontal, diagonally upper right, and diagonally lower left directions are the attention directions, the weight coefficient is set in advance for each direction. If the pixel values at the attention positions in three directions of vertical, horizontal, and diagonally upper right directions are more than or equal to the adjacent pixel values, the extraction function 150c weights the pixel value at the attention position by the weight coefficient determined for each of the three directions (weight coefficient is multiplied by pixel value). As a result, the pixel value at the attention position is changed and emphasized based on the result of such comparison and the weight coefficient determined for each direction.

Subsequently, the output function 150d performs a process of outputting third ultrasonic data 33 on the basis of the continuously extracted ultrasonic data 32 (extraction data) representing the object at step S500 (step S600). In one example, the output function 150d performs a process of outputting the third ultrasonic data 33 by performing an addition process on the ultrasonic data 32 extracted for each parameter x. In another example, the output function 150d outputs the third ultrasonic data 33 by simply adding up the ultrasonic data 32 extracted for each parameter x. In still another example, the output function 150d generates the third ultrasonic data 33 from the ultrasonic data 32 with weighted addition dependent on the value of the parameter x.

The embodiment is not limited to this, and as another example, the output function 150d may generate the synthesis data or correction data as the third ultrasonic data 33. In one example, the output function 150d may generate the third ultrasonic data 33 by synthesizing the data obtained by adding the pieces of second ultrasonic data 31 with respect to the parameter x or the data obtained by adding the pieces of first ultrasonic data 30 with respect to the time t, and the data obtained by adding the ultrasonic data 32 with respect to the parameter x. In another example, the output function 150d may generate the third ultrasonic data 33 by correcting the data obtained by adding the pieces of second ultrasonic data 31 with respect to the parameter x or the data obtained by adding the pieces of first ultrasonic data 30 with respect to the time t, based on the ultrasonic data 32. The embodiment is not limited to this, and as another example, the output function 150d may, for example, perform inverse transformation of the transformation at step S400 on each piece of the ultrasonic data 32 extracted for each parameter x, and then perform an addition process on the inversely transformed data.

As described above, the medical information processing device 100 according to the embodiment applies a transformation to the pieces of first ultrasonic data 30 to generate the pieces of second ultrasonic data 31 characterized by the parameter x other than the time t, which are then used to perform signal extraction. Thus, for example, separating the blood flow image into the characteristic-separated images makes the efficient signal extraction possible.

### Second Embodiment

In the case described in the first embodiment, the transformation function 150b performs Fourier transformation at step S400 to generate the pieces of second ultrasonic data, based on the pieces of first ultrasonic data 30. However, the embodiment is not limited to this. The transformation performed at step S400 is not limited to the Fourier transformation, and more generally may be an expansion process using an orthogonal base.

The process from step S100 to step S300 in the second embodiment is similar to that in the first embodiment. At step S400, in the second embodiment, the processing circuit 150 causes the transformation function 150b to apply a transformation to the pieces of first ultrasonic data 30 and generate the pieces of second ultrasonic data 31 characterized by the parameter other than the time t, which is similar to the first embodiment. Here, in the second embodiment, at step S400, the processing circuit 150 causes the transformation function 150b to generate the pieces of second ultrasonic data 31 by expanding the pieces of first ultrasonic data 30 with an orthogonal base to obtain the expansion coefficient.

Thus, for example, the blood flow image can be separated into the characteristic-separated images classified by the orthogonal base system. As an example, when the orthogonal base system is an orthogonal polynomial, the second ultrasonic data in the case where the parameter x other than the time t is the lowest will be a characteristic-separated image where an oscillation component of a fundamental mode is extracted, and the second ultrasonic data in the case where the parameter x other than the time t is higher will be a characteristic-separated image where a higher-order oscillation component is extracted.

When it is assumed that φ be the orthonormal base system normalized by Expression (3), the pieces of first ultrasonic data 30 be expressed as F(t) as the function of the time t, the second ultrasonic data 31 be expressed as f(x) as the function of the parameter x other than the time t, and N represent the number of frames, F(t) is expanded by the following Expression (4) using f(x).

$$\int dt {φ}^{⋆} ( x , t ) φ ( {x}^{′} , t ) = δ ( x , {x}^{′} ) \qquad ( 3 )$$

$$F ( t ) = \int d x f ( x ) φ ( x , t ) \qquad ( 4 )$$

In this case, f(x) can be calculated using F(t) as shown in Expression (5) below.

$$f ( x ) = \int dtF ( t ) {φ}^{⋆} ( x , t ) \qquad ( 5 )$$

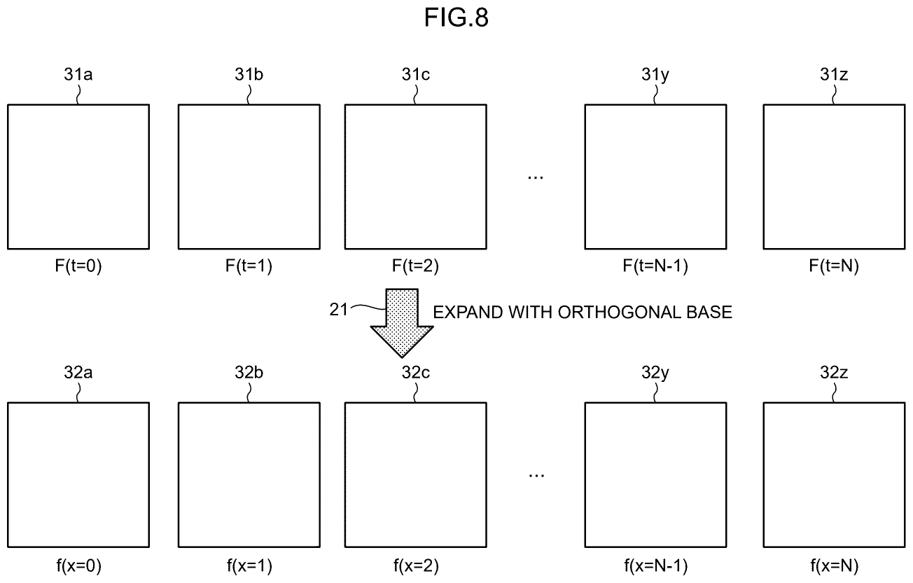

That is, as illustrated in FIG. 8, the processing circuit 150 causes the transformation function 150b to perform a process 21 of expansion using the orthogonal base system on the first ultrasonic data 31a, 31b, 31c, . . . , 31y, 31z to calculate the second ultrasonic data 32a, 32b, 32c, . . . , 32y, 32z using Expression (5). Here, the pieces of first ultrasonic data 31a, 31b, 31c, 31y, and 31z are the data in which the time t corresponds to t=0, t=1, t=2, t=N−1, and t=N, respectively, and the pieces of second ultrasonic data 32a, 32b, 32c, 32y, and 32z are the data in which the parameter x that is other than the time t corresponds to x=0, x=1, x=2, x=N−1, and x=N, respectively.

Examples of the orthonormal base system used here include Legendre polynomials, Chebyshev polynomials, Laguerre polynomials, and Bessel functions. The case where a trigonometric function is used as the orthonormal base system corresponds to the case where the Fourier transformation described in the first embodiment is used. Note that the domain of the integrand of these orthonormal base systems is different depending on the function system. In order to suitably expand the function with such orthonormal base systems, the transformation function 150b may perform the expansion with the orthonormal base systems after transforming the domain of the integrand by the variable transformation as appropriate. If the orthogonal base system is not normalized, the similar process can be performed by performing normalization as appropriate.

At step S500, the extraction function 150c of the processing circuit 150 performs the process of continuously extracting the signal components representing the object from the pieces of second ultrasonic data 31 generated at step S400, for each parameter x other than the time t, using the kernel similar to that used in the first embodiment.

Subsequently, at step S600, the output function 150d performs a process of outputting the third ultrasonic data 33 on the basis of the continuously extracted ultrasonic data 32 (extraction data) representing the object at step S500. In one example, the output function 150d performs the process of outputting the third ultrasonic data 33 by performing an addition process on the ultrasonic data 32 extracted for each parameter x. In another example, the output function 150d outputs the third ultrasonic data 33 by simply adding up the ultrasonic data 32 extracted for each parameter x. In still another example, the output function 150d generates the third ultrasonic data 33 from the ultrasonic data 32 with weighted addition dependent on the value of the parameter x. The embodiment is not limited to this, and as another example, the output function 150d may, for example, perform inverse transformation of the transformation at step S400 on each piece of the ultrasonic data 32 extracted for each parameter x, and then perform an addition process on the inversely transformed data.

As above, the second embodiment has described the case, in a more general form, of generating the pieces of second ultrasonic data on the basis of the pieces of first ultrasonic data 30. In the embodiments, various transformations other than the Fourier transformation are applicable.

### OTHER EMBODIMENTS

Note that the embodiment is not limited by the above examples, and various changes can be made without departing from the gist thereof.

In the embodiments described above, each of the pieces of first ultrasonic data 30 is, for example, two-dimensional or three-dimensional data concerning a pixel value; however, the embodiments are not limited to this. In one example, the transformation function 150b may apply the Fourier transformation not only in the time t direction but also in the spatial direction, and perform the process in the first or second embodiment using the pieces of first ultrasonic data that are displayed in frequency in the spatial direction as well. More generally, the transformation function 150b may perform the process in the first or second embodiment by performing the expansion in the spatial direction as well as in the time t direction, for example, with the orthonormal base such as Legendre polynomials, Bessel functions, spherical harmonic functions, or spherical Bessel functions, and using the pieces of first ultrasonic data displayed using the expansion coefficient.

In the above embodiment, the extraction function 150c performs the process of continuously extracting the signal components representing the object from the first ultrasonic data by the kernel 40 illustrated in FIG. 7, for example, but may alternatively perform a process of extracting the signal components representing the object without using a kernel. For example, the extraction function 150c may input the second ultrasonic data 31 to the trained model that, upon input of a single piece of ultrasonic data with a higher SN ratio than those of the pieces of frame data that are obtained by the ultrasonic scan and that are continuous in the time direction, continuously outputs the signal components representing the object included in the ultrasonic data, and acquire the continuously extracted ultrasonic data 32 representing the object from the trained model. This trained model is trained by using, for example, a dataset in which the ultrasonic data with the improved SN ratio is the input data and the signal components that continuously represent the object in the ultrasonic data are the teacher data. The extraction function 150c inputs the second ultrasonic data to the trained model and acquires the signal components that are output from the trained model and that continuously represent the object. Thus, the signal components representing the object can be continuously extracted from the second ultrasonic data 31 without using a kernel.

According to at least one embodiment described above, the ultrasonic data with high responsiveness and high resolution can be obtained.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device comprising a processing circuit configured to:
acquire a plurality of pieces of first ultrasonic data obtained based on a result of an ultrasonic scan of a subject;
apply a transformation to the pieces of first ultrasonic data to generate a plurality of pieces of second ultrasonic data characterized by a parameter other than time;
continuously extract a signal component representing an object from the pieces of second ultrasonic data; and
output third ultrasonic data, based on the continuously extracted signal component representing the object.

2. The medical information processing device according to claim 1, wherein the parameter is frequency.

3. The medical information processing device according to claim 1, wherein the transformation is Fourier transformation.

4. The medical information processing device according to claim 1, wherein the processing circuit is configured to generate the pieces of second ultrasonic data by expanding the pieces of first ultrasonic data with an orthogonal base to obtain an expansion coefficient.

5. The medical information processing device according to claim 1, wherein the object includes at least one of blood, an in-body tissue, and a contrast medium.

6. An ultrasonic diagnosis device comprising a processing circuit configured to:
cause an ultrasonic probe to perform an ultrasonic scan of a subject;
acquire a plurality of pieces of first ultrasonic data obtained based on a result of the ultrasonic scan;
apply a transformation to the pieces of first ultrasonic data to generate a plurality of pieces of second ultrasonic data characterized by a parameter other than time;
continuously extract a signal component representing an object from the pieces of second ultrasonic data; and
output third ultrasonic data, based on the continuously extracted signal component representing the object.

7. A non-transitory computer readable medium comprising instructions that cause a computer to execute:
acquire a plurality of pieces of first ultrasonic data obtained based on a result of an ultrasonic scan of a subject;
apply a transformation to the pieces of first ultrasonic data to generate a plurality of pieces of second ultrasonic data characterized by a parameter other than time;
continuously extract a signal component representing an object from the pieces of second ultrasonic data; and
output third ultrasonic data, based on the continuously extracted signal component representing the object.
